(19) United States
(12) Patent Application Publication
(10) Pub. No.: US 2022/0287155 A1
(43) Pub. Date: Sep. 8, 2022

(54) RESISTIVE HEATING REACTORS FOR HIGH TEMPERATURE CO2 UPGRADING

(71) Applicant: Lawrence Livermore National Security, LLC, Livermore, CA (US)

(72) Inventors: Simon Hoching Pang, Fremont, CA (US); Jeffery J. Haslam, Livermore, CA (US); James Patrick Kelly, San Francisco, CA (US); Joshuah K. Stolaroff, Oakland, CA (US)

(21) Appl. No.: 17/193,937

(22) Filed: Mar. 5, 2021

(51) Int. Cl. H05B 3/14; B33Y 80/00; B33Y 40/20; B01J 35/02; B01J 19/00

(52) U.S. Cl. CPC H05B 3/141; B33Y 80/00; B33Y 40/20; B01J 35/023; B01J 19/0053; B01J 35/026; B33Y 70/00

(57) ABSTRACT

A product includes a three-dimensional resistive heating element formed by additive manufacturing and a catalytic component on at least an external surface of the resistive heating element. The resistive heating element has a pre-defined geometric arrangement of features, and the resistive heating element includes a conductive ceramic material.

This invention was made with Government support under Contract No. DE-AC52-07NA27344 awarded by the United States Department of Energy. The Government has certain rights in the invention.

### FIELD OF THE INVENTION

The present invention relates to resistive heating reactors, and more particularly, this invention relates to resistive heating reactors for high temperature CO₂ upgrading, and methods of making same.

### BACKGROUND

Production of carbon monoxide (CO) is desirable for the production of fuels and chemicals from a waste carbon dioxide (CO₂) containing feedstock. However, there are technical challenges in converting CO₂ into reduced forms of carbon, such as CO. Reactions for upgrading carbon dioxide (CO₂) from waste feedstock into carbon monoxide (CO) for use in synthesis of liquid fuels or other chemicals typically involve temperatures greater than 700° C. to achieve high equilibrium conversion. Heat is typically provided externally, by burning natural gas to reach the high temperatures thereby, unfortunately, creating additional CO₂. Thus, conventional conversion processes defeat the purpose of capture and conversion of CO₂.

As alluded to above, a major limitation of the conventional process is the use of non-sustainable fossil resources, e.g., natural gas. In sharp contrast, renewable electricity has growing availability and the cost is becoming more affordable. The use of fossil fuels to provide external heat is a primary challenge of these conventional conversion processes since the use of fossil fuels leads to more CO₂ production.

Moreover, in conventional systems, heat is provided generally to the whole reactor, rather than specifically at the site of reaction, e.g., the catalyst surface. Heating the whole reactor has several drawbacks. Heating the whole reactor can lead to undesired side reactions. In addition, startup and shut down of the reactor is typically relatively slow, due to a larger thermal mass that must be heated (e.g., the entire reactor volume). Moreover, the temperature profile tends to be inhomogeneous throughout the reactor thereby causing a deleterious effect on reactor performance (e.g., in terms of reaction rate, reaction selectivity, etc.).

Conventional conversion systems include the presence of catalytic components, such as catalyst powders. In the conventional system, the catalyst powders are used directly in a reactor and the reactor is typically configured as a fluidized bed or a packed bed to promote mixing between the catalyst and the reactants. However, this configuration has an energy penalty associated with overcoming the pressure drop of the reactor. Alternatively, to overcome the issue of the pressure drop, catalyst powders may be coated onto inert extrudates or formed into pellets using binders. However, each of these methods has significant drawbacks. For instance, the coating or forming of pellets introduces additional mass into the reactor and this additional mass must be heated. Moreover, the methods for coating and forming pellets must be controlled precisely to prevent introduction of mass transport barriers to the catalyst surface. Furthermore, heat transfer through these materials is challenging, leading to inhomogeneity in the temperature profile. Packed beds containing catalysts on pellets will generally require non-negligible amounts of pressure to move reactants and products through the reactor.

Another drawback of the conventional conversion system that relies on fossil fuels is maintaining efficiency in the presence of combustion products from the fossil fuel power source. For example, performance of a catalytic conversion system using heat generated with fossil fuels depends on costly mitigations or otherwise separating the combustion gas from the catalyst surface to avoid fouling, contamination, decreased effectiveness, etc. caused by combustion products such as ash, soot, gas species (e.g., sulfur), etc. that tend to diminish the catalyst performance.

Alternatively, in some systems, the heat from fossil fuels is provided indirectly through the external walls of the catalytic reactor to avoid mixing the combustion gases with the reactants, catalysts, and products, but this leads to inhomogeneity in the temperature profile through the reactor.

It would be desirable to provide heat locally to the catalyst, thereby minimizing the amount of energy needed to provide the requisite heat. This may also enable use of renewable electricity for improving the climate impact of the overall process. In this way, utilizing localized heating may improve the energy efficiency of the overall process and lower the carbon intensity of the final product. Even if fossil fuels are used to provide a portion of the energy, it would be desirable to reduce carbon intensity by improving energy efficiency. Moreover, a similar approach of a system providing heat locally to a catalyst may be useful for other chemical production processes, such as production of ammonia, reforming of hydrocarbons, etc.

### SUMMARY

In one embodiment, a product includes a three-dimensional resistive heating element formed by additive manufacturing and a catalytic component on at least an external surface of the resistive heating element. The resistive heating element has a pre-defined geometric arrangement of features, and the resistive heating element includes a conductive ceramic material.

In another embodiment, a method of forming a product for high temperature conversion of one or more reactants includes fabricating a resistive heating reactor using, at least in part, additive manufacturing, where the resistive heating reactor includes a conductive ceramic material and a catalytic component.

In yet another embodiment, a method of using a ceramic resistive heating reactor for converting one or more reactants includes applying a current to the ceramic resistive heating reactor for heating the ceramic resistive heating reactor to a pre-defined temperature, where the ceramic resistive heating reactor comprises a catalytic component. The method includes contacting the one or more reactants with the catalytic component for causing a reaction to form at least one product by flowing the one or more reactants across the heated ceramic resistive heating reactor and collecting the at least one product.

Other aspects and advantages of the present invention will become apparent from the following detailed description, which, when taken in conjunction with the drawings, illustrate by way of example the principles of the invention.

### BRIEF DESCRIPTION OF THE DRAWINGS

FIG. 1A is a schematic drawing of a resistive heating reactor, according to one embodiment.

FIG. 1B is a schematic drawing of a resistive heating reactor having a layer of a catalytic component, according to one embodiment.

FIG. 1C is a schematic drawing of a resistive heating reactor having a series of continuous channels, according to one embodiment.

FIG. 1D is a schematic drawing of a resistive heating reactor having a three-dimensional structure in the form of a gyroid, according to one embodiment.

FIGS. 2A-2D are a series of flow charts of a method of forming a resistive heating reactor, according to one embodiment.

FIG. 3 is a flow chart of a method of using a resistive heating reactor, according to one embodiment.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified.

For the purposes of this application, room temperature is defined as in a range of about 20° C. to about 25° C.

As also used herein, the term “about” denotes an interval of accuracy that ensures the technical effect of the feature in question. In various approaches, the term “about” when combined with a value, refers to plus and minus 10% of the reference value. For example, a thickness of about 10 nm refers to a thickness of 10 nm±1 nm, a temperature of about 50° C. refers to a temperature of 50° C.±5° C., etc.

It is also noted that, as used in the specification and the appended claims, wt % is defined as the percentage of weight of a particular component is to the total weight/mass of the mixture. Vol % is defined as the percentage of volume of a particular compound to the total volume of the mixture or compound. Mol % is defined as the percentage of moles of a particular component to the total moles of the mixture or compound. Atomic % (at %) is defined as a percentage of one type of atom relative to the total number of atoms of a compound.

Unless expressly defined otherwise herein, each component listed in a particular approach may be present in an effective amount. An effective amount of a component means that enough of the component is present to result in a discernable change in a target characteristic of the ink, printed structure, and/or final product in which the component is present, and preferably results in a change of the characteristic to within a desired range. One skilled in the art, now armed with the teachings herein, would be able to readily determine an effective amount of a particular component without having to resort to undue experimentation.

The present disclosure includes several descriptions of exemplary “inks” used in an additive manufacturing process to form the inventive concepts described herein. It should be understood that “inks” (and singular forms thereof) may be used interchangeably and refer to a composition of matter dispersed throughout a liquid phase such that the composition of matter may be “written,” extruded, printed, or otherwise deposited to form a layer that substantially retains its as-deposited geometry and shape with perhaps some, but preferably not excessive, sagging, slumping, or other deformation, even when deposited onto other layers of ink, and/or when other layers of ink are deposited onto the layer. As such, skilled artisans will understand the presently described inks to exhibit appropriate rheological properties to allow the formation of monolithic structures via deposition of multiple layers of the ink (or in some cases multiple inks with different compositions) in sequence.

The following description discloses several preferred structures formed via direct ink writing (DIW), extrusion freeform fabrication, binder-jet fabrication with powders, powder-bed laser fusion, or other equivalent techniques and therefore exhibit unique structural and compositional characteristics conveyed via the precise control allowed by such techniques. The physical characteristics a structure formed by DIW may include having lower layers of the structure are slightly flattened, slightly disfigured from original extrusion, etc. by weight of upper layers of structure, due to gravity, due to nozzle offset height, etc. The three-dimensional structure formed by DIW may have a single continuous filament, ligament, strand, feature, etc. that makes up at least two layers of the 3D structure.

The following description discloses several preferred embodiments of a ceramic resistive heating reactor and/or related systems and methods.

In one general embodiment, a product includes a three-dimensional resistive heating element formed by additive manufacturing and a catalytic component on at least an external surface of the resistive heating element. The resistive heating element has a pre-defined geometric arrangement of features, and the resistive heating element includes a conductive ceramic material.

In another general embodiment, a method of forming a product for high temperature conversion of one or more reactants includes fabricating a resistive heating reactor using, at least in part, additive manufacturing, where the resistive heating reactor includes a conductive ceramic material and a catalytic component.

In yet another general embodiment, a method of using a ceramic resistive heating reactor for converting one or more reactants includes applying a current to the ceramic resistive heating reactor for heating the ceramic resistive heating reactor to a pre-defined temperature, where the ceramic resistive heating reactor comprises a catalytic component. The method includes contacting the one or more reactants with the catalytic component for causing a reaction to form at least one product by flowing the one or more reactants across the heated ceramic resistive heating reactor and collecting the at least one product.

A list of acronyms used in the description is provided below.

3D three-dimensional

B boron

C Celsius

CeO₂ cerium oxide

CH₄ methane

CO carbon monoxide

CO₂ carbon dioxide

DIW direct ink writing

LaCrO₃ lanthanum chromite

ms millisecond

MoSi₂ molybdenum disilicide

nm nanometer

SiC silicon carbide

Si₃N₄ silicon nitride

μm micron

wt % weight percent

ZrB₂ zirconium diboride

Resistive heating (also known as Joule heating) uses electricity to generate heat. According to one embodiment, a resistive heating element may be used in upgrading CO₂ by providing localized heat to drive a desired reaction. In one approach, catalytic elements may be incorporated directly onto a resistive heater. In another approach, a resistive heating element may include an integrated heater/catalyst material to promote local heating at the catalyst surface for contacting reactants and promoting the formation of desired products. In another approach, the resistive heating element may also be made from a catalytic material. In another approach, after startup of the reactor, sustained heating might be provided or partially provided by exothermic reactions enabled by the catalyst.

Conventional resistive heating (also known as Joule heating, resistance heating, or Ohmic heating), is a process of passing an electrical current through a conductive material to produce heat, e.g., a material becomes heated when an electrical current is passed through the material. In some approaches, high temperature heating may be achieved by using electricity. Preferably, a renewable source of the electricity, such as solar, wind, etc., may allow a heating process to be carbon neutral.

Additionally, the conversion reactions of CO₂ are performed with a catalytic component. In preferred approaches, performing the reaction over catalytically active components (e.g., catalytic components) such as metal nanoparticles or reducible metal oxides increases the rate of reaction and reduces the reactor size required to achieve equilibrium.

The novelty of the invention is the integration of the two components into a single reactor for the reactions of interest, and the manufacturing process that leverages LLNL expertise in additive manufacturing and 3D printing of metals, oxides, and ceramics.

As described herein, an embodiment overcomes these limitations by using additive manufacturing techniques to incorporate catalytic components directly on and/or in a resistive heating element thereby providing heat locally at the catalyst region that is critical for CO₂ conversion reactions. An advantage of the described system includes a more efficient use of the energy by having the material of the resistive heating element be the source of heat.

In a resistive heating system, the rate of heating is measured in terms of Power P, such that,

P=I²·R Equation 1

Thus, the rate of resistive heating P is proportional to the square of the electrical current I, and the resistance R of the material, element, etc. of the resistive heating structure, where the current I passes through the R of the material, element, etc. Preferably, the material of the resistive heating element has a high melting point (e.g., above the expected operating temperature range), is free from oxidation and other degradation reactions in the application or degradation reactions can be readily reversed, is not negatively impacted by the conditions of the reaction in the application (e.g., by the reactants), has high tensile strength, has sufficiently low electrical resistivity, has sufficient thermal conductivity to avoid an excessive thermal gradient, has a low temperature coefficient of resistance, and has a suitably low thermal expansion coefficient.

Heating the resistive heating element with a current reduces the time for reactor startup and shutdown, since the heating is focused at the resistive heating element without the limitation of a rate of heat transfer throughout a conventional reactor. The extent of undesired side reactions may be reduced, and overall reactor performance may be improved. The overall energy efficiency of the process may also be improved in this way.

According to one embodiment, a reactor material uses a resistive heating material as a support for a catalytic component. The resistive heating material preferably is a ceramic-based material that is electrically conductive. Moreover, by integrating a resistive heating material and a catalytic component together and passing an electrical current through the integrated material, heat can be provided locally to the catalyst surface, allowing efficient conversion of electricity into heat, and subsequently into chemical energy (e.g., by converting CO₂ into CO). Alternatively, after the reactor having the electrical heating element is fabricated, the catalytic material may be deposited on an exposed surface internal to the fabricated reactor by wet impregnation; deposition from a slurry, fluidized, etc. particle bed, particle flow, etc.; chemical deposition from a vapor (e.g., by Chemical Vapor Deposition, Atomic Layer Deposition, etc.); etc.

According to various embodiments described herein, additive manufacturing techniques may be utilized to create reactors with integrated catalytic and resistive heating elements to provide local electrical heating to the catalyst surface. These factors potentially allow the reactors to be intensified, leading to smaller systems and lower capital costs.

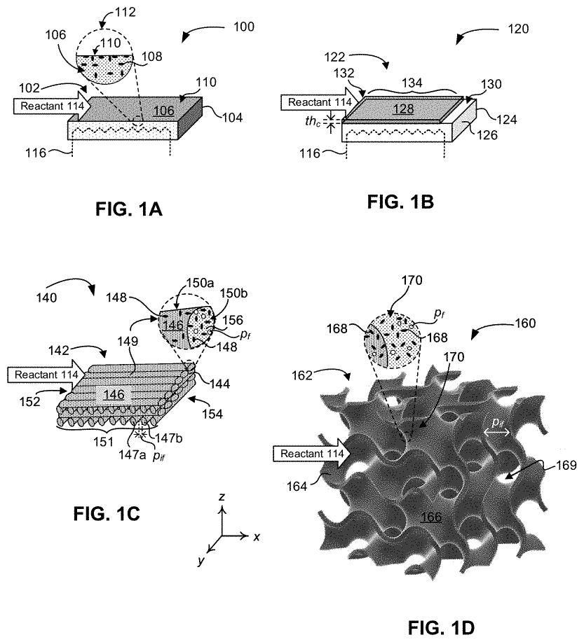

In FIGS. 1A-1D, each schematic diagram depicts a perspective of a portion of products 100, 120, 140, 160, respectively, in accordance with one aspect of an inventive concept. As an option, the present products 100, 120, 140, 160, respectively may be implemented in conjunction with features from any other inventive concept listed herein, such as those described with reference to the other FIGS. Of course, however, each product 100, 120, 140, 160, respectively, and others presented herein may be used in various applications and/or in permutations which may or may not be specifically described in the illustrative embodiments listed herein. Further, each product 100, 120, 140, 160, respectively, presented herein may be used in any desired environment.

As illustrated in FIG. 1A, according to one embodiment, a product 100 includes a resistive heating reactor 102 having a three-dimensional (3D) resistive heating element 104 formed by additive manufacturing. The resistive heating element 104 may have a pre-defined geometric arrangement of features. The resistive heating element 104 includes a conductive ceramic material 106. In one approach, the resistive heating element 104 includes an electrically conductive ceramic material 106. It may be critical for the conductive ceramic material to be electrically conductive for a current to flow through the material and provide resistive heating. In one approach, the conductive ceramic material may be a good thermal conductor to aid in uniform heat transfer and to avoid localized hot spots. In addition, as illustrated in the expanded view 112 of a portion of the resistive heating reactor 102 includes a catalytic component 108 on at least an external surface 110 of the resistive heating element 104. In one approach, the catalytic component 108 is integrated into the conductive ceramic material 106 at a concentration where the catalytic component 108 is present on an external surface 110 of the resistive heating element 104, as illustrated in the expanded view 112 of a portion of the resistive heating element 104. In one approach, the catalytic component 108 is integrated into the resistive heating element 104.

In one approach, the material of the resistive heating element may have catalytic properties. In one approach, a resistive heating element comprising a conductive ceramic material that also performs as a catalytic component may not have an additional different catalytic component added to the resistive heating element. For example, a resistive heating element includes an electrically conductive material that also serves as a catalyst for CO₂ upgrading, e.g., molybdenum carbide. In another approach, a resistive heating element comprising a conductive ceramic material that performs as a catalytic component may also have a coating on the surface of a different catalytic component.

According to various embodiments, the conductive ceramic material may include semiconducting materials commonly used in conventional resistive heating elements. In some approaches, the conductive ceramic material may include a metal carbide, a metalloid carbide (e.g., silicon carbide (SiC), etc.), a metal boride, a metalloid boride, a metal nitride, a metalloid nitride (e.g., silicon nitride (Si₃N₄), etc.), a metal oxide, a metalloid oxide, a metal silicide, and/or other materials that would become apparent to one skilled in the art upon reading the present disclosure. In some approaches, the conductive ceramic material of the resistive heating element may include a combination of electrically conductive ceramic materials, electrically insulating ceramic materials, etc.

Preferably, the electrically conductive ceramic material is stable against degradation at high temperature in a steam/reducing environment of the reactor. In one approach, the material may produce a protective glass coating in an air environment, e.g., SiC, molybdenum disilicide (MoSi₂), etc. For example, molybdenum disilicide (MoSi₂) is a well-known electrically conductive furnace element material that is stable for resistive heating in an air environment up to 1800° C. It is known that the formation of a glass (e.g., silicon-based, etc.) may contribute to the protection against degradation in air with a careful selection of compounds (e.g., non-oxides are protected in oxidizing atmospheres). Zirconium diboride (ZrB₂) is another conductive material that has high temperature stability. Some of the catalytic oxides may be able to perform as resistive heating elements as well, depending on the catalytic requirements, as there are oxide materials that have been proven for related applications. In one approach, a resistive heating element may include a conductive ceramic material with a level of resistive heating properties and a coating of a catalytic component that also provides resistive heating elements that may augment the localized heat of the resistive heating element. For example, cerium oxide (CeO₂) is a high temperature oxygen conductor and has been applied to high temperature applications. Lanthanum chromite (LaCrO₃), a perovskite ceramic and semiconductor, may be separately employed as a heating element, catalyst, etc.

In other approaches, the conductive ceramic material may include electrically conductive transition metal compounds. The electrical conductivity may be present in the conductive ceramic material in the form of electrons, electron holes, a result of ionic conductivity, or a combination thereof. For example, the conductive ceramic material may include at least one of the following: a transition metal carbide, a transition metal boride, a transition metal nitride, a transition metal silicide, etc.

In one approach, the conductive ceramic material is in powder form. A powder of conductive ceramic material includes ceramic particles. These powders may be partially densified to provide sufficient electrical conductivity while increasing the surface area for catalytic reactions. In some approaches, partially densified powder material may reduce the economic cost (time, temperature, etc.) for the fabrication of the electrically heated reactor material. This maybe particularly beneficial if the conductive ceramic is both the electrical heater and the catalytic material. In some approaches, the ceramic particles may have an average diameter in a range of about 10 nanometers (nm) to about 50 microns (μm). In preferred approaches, the ceramic particles may have an average diameter in a range of about 100 nm to 25 μm. In one approach, the powder of conductive ceramic material may include large ceramic particles in a mixture with smaller ceramic particles for various packing density advantages.

In one approach, the conductive ceramic material may include oxide particles, e.g., cerium oxide particles, etc. In one approach, the particle size of the conductive ceramic material may be tailored to optimize the reactivity of the resistive heating element, e.g., roughness, surface area, etc.

In preferred approaches, the conductive ceramic material may be amenable to a powder-based additive manufacturing process. In one approach, the conductive ceramic material has a high surface area for dispersing, incorporating, etc. the catalytic component. The conductive ceramic material provides a support for the catalytic component for optimizing the contacting between the one or more reactants and the catalytic component.

The conductive ceramic material preferably has a high melting point. In one approach, the melting point of the conductive ceramic material is greater than 1800° C. In one approach, the melting point of the conductive ceramic material is greater than 2000° C. In various approaches, the conductive ceramic material may include one of the following: SiC (melting point 2730° C.), MoSi₂ (melting point 2030° C.), ZrB₂ (melting point 3000° C.), CeO₂ (melting point 2400° C.), etc.

In one approach, the resistivity of the material of the resistive heating element (e.g., support, substrate, etc.) may be tailored for a desired application. In one approach, a composition of the conductive ceramic material may be tailored for the pre-defined temperature of a desired reaction. For example, a composition of the ceramic material may include less conductive materials, e.g., less conducting boride, less conducting oxide, etc. mixed with higher conducting ceramic material, e.g., SiC, ZrB₂, etc.

In one approach, a structure may be fabricated with ceramic materials and then the ceramic materials of the structure may be converted to have a resistive heating property using reactive sintering techniques. For example, structure may be fabricated with ZrO₂, and then the fabricated ZrO₂ structure is reduced and reacted with a boron (B) source to convert the fabricated structure material into ZrB₂. For example, ZrO₂ may be reacted with B₄C to form ZrB₂ composites. In another example, ZrO₂ may be reduced by carbothermal reduction using C and then reacted with B to form ZrB₂. In another example, ZrO₂ may be reduced by borothermal reduction and then reacted with B to form ZrB₂. In another example, ZrO₂ may be reduce by magnesiothermic reduction and then reacted with B to form ZrB₂. In another example, Zr metal may be reacted with B. In another example, combinations of these reaction approaches may be conducted.

The catalytic component may be incorporated on a surface of the heat resistive element according to a desired reaction. In preferred approaches, the catalytic component is in the particle form. In one approach, the catalytic component may be metal oxide nanoparticles, for example, particles of reducible metal oxides. In another approach, the catalytic component may be metal nanoparticles. In one approach, an average diameter of the particles may be less than a micron. For example, the catalytic component includes nanoparticles having an average diameter in the nanoscale (e.g., greater than 0 nm to less than 1000 nm).

In one approach, use of an intermediate step may be preferred. For example, oxidation of ZrB₂ may form a porous ZrO₂ oxidation scale filled with a glass phase. Intentional removal of the glass phase, leaving behind a porous zirconia scale may be a beneficial structure to support a catalyst.

In one approach, the catalytic component may be a single metal. In another approach, the catalytic component may include a combination of metals. In one approach, the catalytic component may include a combination of metals and metal oxides. In one approach, the catalytic component may be an alloy. In one approach, the catalytic component may be metallic. In one approach, the catalytic component may be a ceramic material.

In various approaches, the catalytic component may include a metal (e.g., copper, nickel, iron, platinum, palladium, rhodium, silver, etc.), and/or a combination of metals. In some approaches, the catalytic component may include a metal oxide (e.g., Al₂O₃, SiO₂, etc.), a reducible metal oxide (e.g., CeO₂, NiO, etc.), a metal carbide, etc. In some approaches, the catalytic component may include a transition metal, a transition metal oxide (e.g., vanadium oxide, titanium oxide, cerium oxide, zirconium oxide, etc.), a transition metal carbide, etc. In other approaches, the catalytic component may include a zeolite, a perovskite (e.g., LaNiO₃, LaₓSr₁₋ₓNiO₃ (where x is between 0 and 1), LaNiₓFe₁₋ₓO₃ (where x is between 0 and 1), LaNiₓMn₁₋ₓO₃ (where x is between 0 and 1), LaMnO₃, SrMnO₃, etc.), metal-organic frameworks, etc. In some approaches, the catalytic component may include a combination of any of the catalytic component materials described herein.

For example, for a reverse water-gas shift reaction, the catalyst component includes copper and/or cerium oxide, titanium oxide, molybdenum carbide, etc. For another example, a dry reforming of methane reaction, the catalyst component includes nickel, zirconium oxide, perovskite catalysts, molybdenum carbide catalysts, etc.

According to one embodiment, the catalytic component is in particle form. In preferred approaches, the catalytic component is in nanoparticle form.

In one approach, the catalytic elements may be incorporated directly onto the resistive heating element during the manufacturing process. In another approach, the catalytic elements may be incorporated directly on the resistive heating element in a post-manufacturing step. In one approach, the catalytic elements are coated onto the support, substrate, resistive heating element, etc. In one approach, the catalytic component may be present only on the surface of the resistive heating element. In one approach, the catalytic component may be present only on a portion of the surface of the resistive heating element, preferably, in a pre-defined location on the surface of the resistive heating element. In one approach, the resistive element may be comprised of a catalytic material.

For example, in an exemplary approach, a resistive heating reactor may include a cerium oxide support having a nickel metal particle as the catalytic component for a desired reaction. The interface between the catalytic component, e.g., nickel particle, and the conductive ceramic support, e.g., cerium oxide, promotes good reactivity for some desired reactions. In another approach, the first catalytic component, e.g., nickel particle, may be proximate to the second catalytic component, e.g., cerium oxide, for providing an interface between the two catalytic components. Both catalytic components may be positioned on a surface of the resistive heating element comprising a conductive ceramic material, e.g., silicon carbide.

In one approach, an amount of the catalytic component may be in a range of 0.1 weight percent (wt. %) to less than about 50 wt. % of total weight of the resistive heating element and the catalytic component.

In preferred approaches, a resistive heating reactor 102 includes a resistive heating element 104 that forms an electrical circuit 116 thereby resulting in resistive heating. As drawn in FIG. 1A, the generation of the electrical circuit 116 indicates the effect of the conductive ceramic material 106 having a suitably low resistance to provide electrical conduction, and suitably high enough to provide resistive heating. In one approach, the resistive heating reactor may not include a wire associated with the electrical circuit 116.

In one approach, the resistive heating reactor may include a conductive wire associated with the electrical circuit 116 to supplement, provide a portion, provide most, provide all of the heating, etc. The conductive wire may be incorporated by one of several means known to those knowledgeable in the art of ceramic processing and furnace element design. In one approach, a wire may be positioned inside a ceramic opening formed by additive manufacturing techniques (e.g., with or without potting by a tube, passageway in a ceramic material such as with a sealant/potting material in many types of electrical furnaces). In some approaches, some conductive ceramic materials may benefit from a boost from a resistive element at low temperatures. The incorporation of a wire would need to consider consequences specific to the particular selected conductive ceramic at elevated temperature which might disrupt the uniformity of heating (e.g., short the current from the conductive ceramic, etc.). In some approaches, including a wire associated with the electrical circuit may be tailored with judicious skill to select materials for the design of an optimized reactor. For example, conductive ceramic materials that may demonstrate a large increase in resistivity with temperature, e.g., platinum, may involve careful tailoring of a resistive heating reactor having a wire associated with the electrical circuit.

According to one embodiment, one or more reactants 114 may be passed across (arrow) the material 106 including the catalytic component 108 for contacting the one or more reactants 114 with the catalytic component 108 at the temperature of the resistive heating element 104 to form a desired product. One or more reactants 114 may be a liquid, a gas, a solid dissolved in a liquid, etc.

In one embodiment, the catalytic component is preferably added as a coating onto the surface of the resistive heating element. For economic reasons, coating defined surface regions of a resistive heating element with the catalytic component may reduce the amount of catalytic component in the resistive heating reactor. For example, incorporating expensive catalytic component materials, e.g., Pt, Rh, etc., into the conductive ceramic material for forming a monolith structure (as illustrated in FIG. 1A) may not be an economically preferred approach.

As illustrated in FIG. 1B, according to one embodiment, a product 120 includes a resistive heating reactor 122 having a 3D resistive heating element 124 formed by additive manufacturing. The resistive heating element 124 may have a pre-defined geometric arrangement of features. The resistive heating element 124 includes a conductive ceramic material 126. The resistive heating reactor 122 includes a catalytic component 128 on at least an external surface 130 of the resistive heating element 124. In one approach, the catalytic component 128 may be a coating 132 on only a portion 134 of a surface 130 of the resistive heating element 124. In one approach, the catalytic component may be a coating on the entire surface of the resistive heating element.

In some approaches, a thickness thₑ of the coating 132 may be in a range of greater than 0 nm and less than about 50 μm. In a preferred approach, a thickness thₑ of the coating 132 may be in a range of greater than 0 nm and less than about 500 nm.

In one approach, the coating 132 on only a portion 134 of the surface 130 of the resistive heating element 124 may be at a pre-defined location. For example, the pre-defined location may be adjacent the path of a flow of the reactant to enable targeted contacting of the reactant with the catalytic component at a high temperature.

In preferred approaches, a resistive heating reactor 122 includes a resistive heating element 124 that forms an electrical circuit 116 thereby resulting in resistive heating. As drawn in FIG. 1B, the generation of the electrical circuit 116 indicates the effect of the conductive ceramic material 126 having a suitably low resistance to provide electrical conduction, and suitably high enough to provide resistive heating.

One or more reactants 114 may be passed across (arrow) the coating 132 of catalytic component 128 on the resistive heating element 124 for contacting the one or more reactants 114 with the catalytic component 128 at the temperature of the resistive heating element 124 to form a desired product.

In one embodiment, selected additive manufacturing processes allow the formation of a 3D structure having pre-defined porosity for increasing the catalytically active surface area of the reactor. In one approach, as illustrated in FIG. 1C, a product 140 includes a resistive heating reactor 142 having a 3D resistive heating element 144 formed by additive manufacturing. The resistive heating element 144 may have a pre-defined geometric arrangement of features. As shown in FIG. 1C, for example, a 3D resistive heating element 144 may include a printed conductive ceramic structure having a plurality of layers in which each layer (e.g., a first layer, a second layer, a third layer, etc.) is formed from the at least one filament (e.g., strand, ligament, feature, etc.). In one approach, the filament may be a continuous filament forming all the layers of the structure.

In some approaches, the resistive heating element may have hierarchical porosity, such that the structure printed by additive manufacturing processes has an inter-filament porosity (e.g., between the printed filament) and an intra-filament (e.g., within the material of the printed filament). Inter-filament pores are defined as pores between two adjacent filaments. A structure is comprised of a plurality of filaments and the space between adjacent filaments is the inter-filament space, e.g., inter-filament pores. Intra-filament pores are defined as pores inside the associated filament. Each filament of a structure has intra-filament space comprised of material and pores, e.g., intra-filament pores.

In one approach, the printed filament may have intra-filament porosity that is formed as a result of the additive manufacturing process or a post-printing treatment process. In one approach, the formation of the printed filaments from a composition of conductive ceramic material and a binder, pore-forming agent, etc. using an additive manufacturing process. A post-printing process for removing the binder, pore-forming agent, etc. allows the formation an intra-filament pores. In various approaches, intra-filament porosity includes pores having an average diameter smaller than the average diameter of the printed feature, filament, strand, etc. and smaller than the average diameter of the inter-filament pores. The inter-filament pore spaces may be systematically varied by the geometry, creating of hierarchy of porosity in inter-filament spaces in addition to intra-filament spaces.

In some approaches, inter-filament pores may have an average diameter in a range of greater than 10 micrometers to less than 1 centimeter. In one approach, a size of the inter-filament pores may be defined by the additive manufacturing technique. In some approaches, the intra-filament pores may have an average diameter in a range of greater than 0 nm to about 300 μm and may be larger. In some approaches, tuning the intra-filament pores may include a pre-defined passageway, roughness, etc. In preferred approaches, intra-filament pores formed with sacrificial components, e.g., porogen, may have an average diameter greater than 0 nm to less than 100 nm. In one approach, a size of the intra-filament pores may be defined by the particle size of the binder, pore-forming agent, etc.

In a preferred approach, a pre-defined geometric arrangement of features includes an open cell structure having continuous channels 149 through the resistive heating element 144 from one side 152 of the resistive heating element 144 to the other side 154 of the resistive heating element 144. In one approach, the continuous channels may be flow channels for the passage of reactant through the resistive heating element. The continuous channel 149 may be formed from a filament 147a, 147b extruded to form a layer of the structure. The resistive heating element 144 may have a pre-defined geometric arrangement of features (e.g., filaments 147a, 147b) that represents a log-pile structure. In one approach, the filaments 147a, 147b may form inter-filament pores p_{if} between the filaments 147a, 147b within a layer 151 and between adjacent layers.

The resistive heating element 144 includes a conductive ceramic material 146 and a catalytic component 148. As illustrated in the magnified view of a portion of the continuous channel 149, the resistive heating reactor 142 includes a catalytic component 148 on at least an external surface 150a, 150b of the resistive heating element 144. In one approach, an external surface may include an external surface 150a on the outer surface of the resistive heating element 144. In one approach, an external surface 150b on the inside 156 of the continuous channel 149, as illustrated in the expanded view of the continuous channel 149. In any approach, a catalytic component 148 may be present on at least a portion of the surface 150a, 150b, of the resistive heating element 144. In one approach, the ceramic material 146 of the continuous channel 149 may include intra-filament pores p_{f}.

In one approach, the catalytic component 148 may be a coating on only a portion of an external surface 150a of the resistive heating element 144. For example, a coating comprising the catalytic component 148 may be printed, deposited, etc. to a portion of the external surface 150a of the resistive heating element.

In preferred approaches, a resistive heating reactor 142 includes a resistive heating element 144 that forms an electrical circuit in the conductive ceramic material 146 resulting in resistive heating. For example, the conductive ceramic material has a sufficiently low resistance to provide electrical conduction and sufficiently high resistance to provide resistive heating. One or more reactants 114 may be passed across and/or through the continuous channels 149 having a catalytic component 148 on at least a portion of the surface of the continuous channels 149 of the resistive heating element 144 for contacting the one or more reactants 114 with the catalytic component 148 at the temperature of the resistive heating element 144 to form a desired product.

In one embodiment, novel reactor geometries constructed out of triply periodic minimal surface (TPMS) structures increase the geometric surface area and introduce tortuosity into the flow path, leading to better fluid phase mixing. As illustrated in FIG. 1D, a gyroid structure may be formed by additive manufacturing processes. In one approach, a product 160 includes a resistive heating reactor 162 having a 3D resistive heating element 164 formed by additive manufacturing. The resistive heating element 164 may have a pre-defined geometric arrangement of features. As illustrated in FIG. 1D, a 3D resistive heating element 164 may include a printed conductive ceramic structure having an increased geometric surface area and tortuosity representative of a TPMS structure. In one approach, a pre-defined geometric arrangement of features includes an open cell structure having continuous channels 169 through the resistive heating element 164 from one side of the resistive heating element 164 to the other side of the resistive heating element 164. The continuous channels 169 may wind, bend, twist, etc. through the resistive heating element while allowing a continuous flow through the resistive heating element from one side to the other side of the resistive heating element. The other side may be an opposite side of the resistive heating element, the other side may be an adjacent side of the resistive heating element. The continuous channels allow unobstructed flow of one or more reactants through the resistive heating reactor. As illustrated in FIG. 1D, in one approach, the diameter of the hollow region of continuous channels 169 may represent inter-filament pores p_{if} of the structure.

The resistive heating element 164 includes a conductive ceramic material 166 and a catalytic component 168. As illustrated in the magnified view of a portion of an external surface 170 of the continuous channel 169, the resistive heating reactor 162 includes a catalytic component 168 on at least an external surface 170 of the resistive heating element 164. In one approach, the conductive ceramic material 166 may include intra-filament pores p_{f}.

The geometric arrangement of features of the 3D TPMS structure as illustrated in FIG. 1D maximizes continuous channels throughout the structure, where each continuous channel has an external surface facing the flow of the one or more reactants through the continuous channel. As illustrated in the expanded view of the external surface, e.g., reactant-facing-surface, the catalytic component 168 is present at the surface of any continuous channel of the structure. In any approach, a catalytic component 168 may be present on at least a portion of the surface 170 of the resistive heating element 164.

In preferred approaches, a resistive heating reactor 162 includes a resistive heating element 164 that forms an electrical circuit in the conductive ceramic material 166 resulting in resistive heating. For example, the conductive ceramic material has a sufficiently low resistance to provide electrical conduction and sufficiently high resistance to provide resistive heating. One or more reactants 114 may be passed across and/or through the continuous channels 169 having a catalytic component 168 on at least a portion of the surface of the continuous channels 169 of the resistive heating element 164 for contacting the one or more reactants 114 with the catalytic component 168 at the temperature of the resistive heating element 164 to form a desired product.

In a preferred approach, the contacting of the one or more reactants and the catalytic component occurs at the internal continuous channels of the 3D structure, e.g., gyroid, TPMS, a hollow cylinder, a log-pile structure, etc. The entire wall of the internal continuous channel may be heated to the desired temperature thereby allowing rapid heat transfer to the one or more reactants.

According to various embodiments, many reactions may likely benefit from the described types of reactor design. In particular, reactions that create low-carbon intensity fuels, and thus participate in circular carbon economy are desirable. In one approach, a process may include a reverse water-gas shift reaction over copper (Cu) and/or cerium oxide (e.g., CeO₂) or titanium oxide (TiO₂) catalysts as shown in Equation 2:

CO₂+H₂⇄CO+H₂O, ΔH_{298K}⁰=41 kJ/mol Equation 2

In another approach, a process may include the dry reforming of methane (CH₄) as shown in Equation 3, over nickel (Ni) and/or zirconium oxide (ZrO₂), perovskite catalysts, etc.:

CO₂+CH₄⇄2CO+2H₂, ΔH_{298K}⁰=247 kJ/mol Equation 3

In one approach, a Sabatier reaction (i.e., CO₂ methanation reaction), as shown in Equation 4, may be optimized using the apparatus, over a nickel catalyst at temperatures in a range of 300 to 400° C.

CO₂+4H₂⇄CH₄+2H₂O, ΔH_{298K}⁰=—165 kJ/mol Equation 4

In one approach, a reaction for oxygen recovery from CO₂, using high temperatures to convert CO₂ into CO and O₂, as shown in Equation 5.

CO₂⇄CO+½O₂, ΔH_{298K}⁰=283 kJ/mol Equation 5

In one approach, a reaction for oxygen recovery from CO₂ using high temperatures to convert CO₂ into solid carbon (C) and O₂, as shown in Equation 6.

CO₂⇄C+O₂, ΔH_{298K}⁰=394 kJ/mol Equation 6

As described herein, the use of the resistive heating reactor is not limited to CO₂ upgrading. In particular, the use of the heat resistive element may be applied to any reaction where a solid catalyst needs to be supported on another material and heat must be applied. The reactants may be in the gas phase or in the liquid phase, and the reactants may be gases, liquids, or solids.

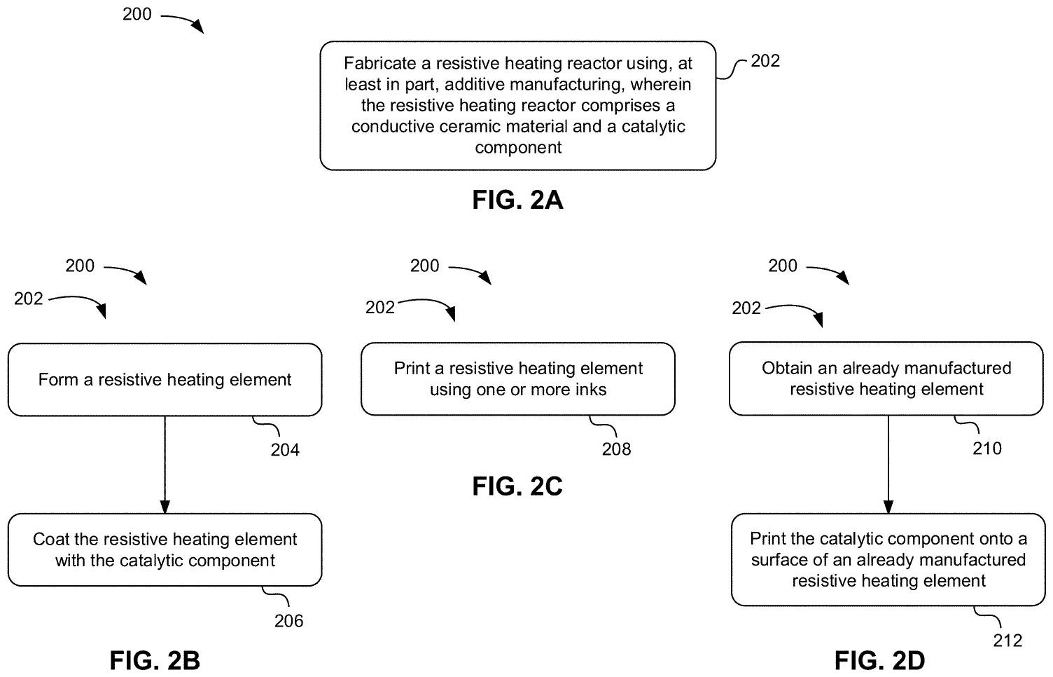

FIGS. 2A-2D show a method 200 for forming a product for high temperature conversion of one or more reactants, in accordance with various aspects of one inventive concept. As an option, the present method 200 may be implemented to construct structures such as those shown in the other FIGS. described herein. Of course, however, this method 200 and others presented herein may be used to form structures for a wide variety of devices and/or purposes which may or may not be related to the illustrative embodiments listed herein. Further, the methods presented herein may be carried out in any desired environment. Moreover, more or less operations than those shown in FIGS. 2A-2D may be included in method 200, according to various embodiments. It should also be noted that any of the aforementioned features may be used in any of the embodiments described in accordance with the various methods.

According to a general embodiment as illustrated in the flow chart of FIG. 2A, method 200 includes operation 202 of fabricating a resistive heating reactor using, at least in part, additive manufacturing. The resistive heating reactor includes a conductive ceramic material and a catalytic component. According to the application of the resistive heating reactor, the fabrication may include several different approaches. Each approach describes fabrication of a resistive heating reactor as described herein.

In one approach of operation 202 of fabricating the resistive heating reactor, depicted in FIG. 2B, operation 202 includes operation 204 of forming a resistive heating element. The resistive heating element may be formed by an additive manufacturing process of forming a monolith, a bulk structure, etc. In one approach, an additive manufacturing process may include powder bed methods where the shape is formed by one of the following: selective deposition and curing of binders followed by subsequent de-binding and sintering, a consolidation processes, etc. In one approach, an additive manufacturing process may include jetting of an ink having ceramic precursors that may be dried, cured etc. before depositing additional material followed by subsequent de-binding and sintering. In one approach, an additive manufacturing approach may include extrusion of an ink, paste, etc. to write a filament followed by subsequent de-binding and sintering. In one approach, the resistive heating element may include a conductive ceramic material. In another approach, the resistive heating element may include a mixture of the conductive ceramic material and the catalytic component.

Operation 206 includes coating the resistive heating element with the catalytic component. The resistive heating element may be coated with the catalytic component by any known technique by one skilled in the art, e.g., wet impregnation, dip coating, slurry coating, spray coating, vapor deposition, atomic layer deposition, electroplating, electroless deposition, deposition of aerosolized particles, etc. In one example, a conductive substrate material may enable coating of the catalytic component by using an electroplating technique.

In various approaches, the coating forms a layer of catalytic component directly on the surface of the resistive heating element. The thickness of the layer may be in a range of greater than 0 nm to less than 50 μm. Preferably, the thickness of the layer may be in a range of greater than 0 nm to less than about 500 nm. In one approach, a portion of the coating may include a catalyst, for example, the coating may include a non-catalytic ceramic support with the catalyst dispersed on the surfaces of the support material.

In one approach, operation 206 may include coating an already manufactured resistive heating element with the catalytic component. The resistive heating element may be pre-printed, molded, etc.

In a second approach of operation 202, as depicted in FIG. 2C, operation 208 includes printing a resistive heating element using one or more inks. In one approach, the printing may include a mixed ink, where the components of the ink, e.g., the conductive ceramic material, catalytic component, etc., are mixed before the printing of the resistive heating element. In various approaches, the mixture may be combined at room temperature. In some instances, temperature may vary as determined by the component of the mixture as generally understood by one skilled in the art.

In one approach, the components of the ink may be mixed in the nozzle, optionally with assistance from a mixer, as the ink is being extruded from the nozzle. For example, in one approach, a gradient may be created in the density, amount, etc. of catalyst along one or more dimensions of the structure. Alternatively, in one approach, a uniform density of catalyst may be printed on pre-defined regions of the structure. The mixer rate of the ink in a mixing system may be defined as the rate of mixing the ink in a nozzle, cartridge, etc. prior to extruding the ink to a substrate. The rate is measured in revolutions per minute of a paddle, impeller, stirring rod, etc. In one approach, selecting a mixer rate for setting a porosity includes determining the viscosity of the ink, parts of ink, etc.

In another approach, two separate inks are prepared, and then the two inks are co-extruded during the printing of the resistive heating element. In yet another approach, two separate inks are prepared, and then the first ink comprising the conductive ceramic material is extruded to print the resistive heating element, followed by printing the second ink comprising the catalytic component (or vice versa). The second ink may be extruded directly on the surface of the printed resistive heating element so that the catalytic component is a coating on at least a portion of the surface of the resistive heating element.

In a third approach of operation 202, depicted in FIG. 2D, operation 202 includes operation 210 of obtaining an already manufactured resistive heating element comprising a conductive ceramic material. The resistive heating element may be pre-printed, molded, etc.

Operation 212 includes printing the catalytic component onto a surface of the already manufactured resistive heating element.

In various approaches, method 200 of fabricating a resistive heating element includes using additive manufacturing techniques, at least in part, that are highly scalable and compatible to form a three-dimensional (3D) structure (e.g., three-dimensional (3D) printing). Such additive manufacturing techniques include an extrusion-based technique (e.g., direct ink writing (DIW), a powder bed-based technique (e.g., binder jetting, etc.), a material jetting technique, a sheet lamination technique, an electrostatic deposition technique, a laser fusion technique, a mold, a template, etc. In various approaches, the formed 3D structure has physical characteristics of formation by an additive manufacturing technique. In various approaches, physical characteristics may include filaments arranged in a geometric pattern, a patterned outer surface defined by stacking filaments, a defined porosity (e.g., ordered, controlled, non-random, etc.), a porosity having pores with measurable average diameters, an outer surface and/or embedded surface having ridges and/or channels characteristic of a structure formed from printed filaments, etc. Thus, using these additive manufacturing techniques allows engineering of parts and production of optimal geometry for efficient mass transport and mechanical strength.

In some approaches, 3D printing of an ink allows extrusion of a bulk material to print struts in desired geometric patterns. The forming a 3D printed structure may include extruding a mixture of conductive ceramic material and/or catalytic component as an ink through the nozzle during direct ink writing processes, curing the 3D structure and sintering the cured 3D structure.

In some approaches, the 3D printed structure is cured following the printing step to crosslink monomers, oligomers, polymers, and/or a combination thereof. In various approaches, the curing step may be tuned according to the binder included in the ink. In various approaches, the type of binder material may include one or more of the following: a thermal curing binder, e.g., phenolic reaction compounds; a chemical curing binding, e.g., epoxies; UV curing binders, e.g., acrylates; non-ionic surfactants, e.g., poly(ethyleneglycol)-block-poly(propyleneglycol); polyethylenimine; polyvinyl alcohol; polyvinylpyrrolidone; waxes; and a large variety of binders for powder processing compatible with aqueous or non-aqueous processing, etc. Curing conditions may be adjusted for a particular binder material and the curing process known to one skilled in the art of powder based printing of additively manufactured components. As an example, the curing step includes heating the printed structure at a temperature up to about 150° C. for a duration of time. In an exemplary approach, the curing includes heating the structure to 150° C. for 16 hours.

In various approaches, the cured 3D structure may be de-binded and sintered. De-binding and sintering may occur as separate thermal treatments, in a combined thermal treatment, etc. De-binding typically occurs at temperatures ranging from 200 to 600° C. for decomposing polymers, removing polymers, etc. from the structure. Sintering typically occurs at temperatures above 1000° C., and as high as 2000° C., in inert gas environments (e.g., nitrogen, argon, helium, etc.), reducing gas environments (e.g., forming gas, hydrogen, etc.), oxidizing gas environments (e.g., carbon dioxide, air, oxygen, etc.), or other reactive gas environments (e.g., ammonia, etc.). Temperature and duration of de-binding and sintering may vary for different materials based on the temperature preferred to activate mass transport mechanisms.

According to various embodiments described herein, the conductive ceramic material may include semiconducting materials commonly used in conventional resistive heating elements, e.g., silicon carbide (SiC). Preferably, the conductive ceramic material is stable against degradation at high temperature in a steam/reducing environment of the reactor. The melting point of the conductive ceramic material is preferably greater than 1800° C. In various approaches, the conductive ceramic material may include one or more of the following: SiC, MoSi₂, ZrB₂, CeO₂, etc.

In various approaches to method 200, the catalytic component may include one or more of the following: a metal (e.g., copper, nickel, iron, platinum, palladium, rhodium, silver, etc.), a combination of metals, etc. In some approaches, the catalytic component may include a metal oxide (e.g., Al₂O₃, SiO₂, etc.), a reducible metal oxide (e.g., CeO₂, NiO, etc.), a metal carbide, etc. In some approaches, the catalytic component may include a transition metal, a transition metal oxide (e.g., vanadium oxide, titanium oxide, cerium oxide, zirconium oxide, etc.), a transition metal carbide, etc. In other approaches, the catalytic component may include a zeolite, a perovskite (e.g., LaNiO₃, LaₓSr₁₋ₓNiO₃ (where x is between 0 and 1), LaNiₓFe₁₋ₓO₃ (where x is between 0 and 1), LaNiₓMn₁₋ₓO₃ (where x is between 0 and 1), LaMnO₃, SrMnO₃, etc.), metal-organic frameworks, etc. In some approaches, the catalytic component may include a combination of any of the catalytic component materials described herein.

Reducible metal oxides may exist in multiple oxidation states at various temperatures. For example, Ce may exist as a mixture of 4+ and 3+ oxidation state depending on the environment. In one approach, reducible metal oxide particles may be applied as the catalytic component to the resistive heating element as metal oxide particles, and then be easily reduced to elemental metal particles. Following coating, incorporation, printing, etc. of the reducible metal oxide powder, the positioned metal oxide powder on the resistive heating element may be reduced to form an elemental metal having an increased surface area. For example, nickel oxide may be coated on a resistive heating element, and then the nickel oxide coating may be reduced with a subsequent reducing gas process.

In one approach, decomposition reactions may provide a means to obtain high surface area materials. For example, decomposition of a metal carbonate into a metal oxide often produces a high surface area metal oxide due to the concurrent production of gases during the conversion to the oxide.

According to one embodiment, the catalytic component is in particle form. In preferred approaches, the catalytic component is in nanoparticle form. Catalytic nanoparticles may be coated onto catalytic supports such as microparticles.

In various approaches, formation of the resistive heating reactor may be tuned to the desired reactant, e.g., gas, conversion reaction in a specific application. For example, a resistive heating reactor can be formed having the preferred catalytic component for a reverse water-gas shift reaction, e.g., copper and/or cerium oxide or titanium oxide, at the surface of the conductive ceramic resistive heating element.

In some approaches, the filler additive may include a reinforcing filler for reinforcing the printed structure, e.g., to provide durability to the printed structure. In some approaches, the filler additive may include a non-reinforcing filler for increasing the volume of the ink formulation. Exemplary fillers include fumed silicas, precipitated silicas, diatomaceous earth, calcium carbonate, metal carbides, metal borides, etc.

In approaches, a concentration of filler may be less than about 30 wt %, preferably up to about 20 wt % of total mixture but may be more or less. In various approaches, the concentration of filler may be determined by the filler included in the mixture. In one preferred approach, a filler may have a maximum reinforcement effect at 20 wt %, so then 20 wt % is the desired concentration of the filler in the mixture. In one approach, the filler additive may be removed during post-printing treatment. In another approach, the filler additive may not be removed.

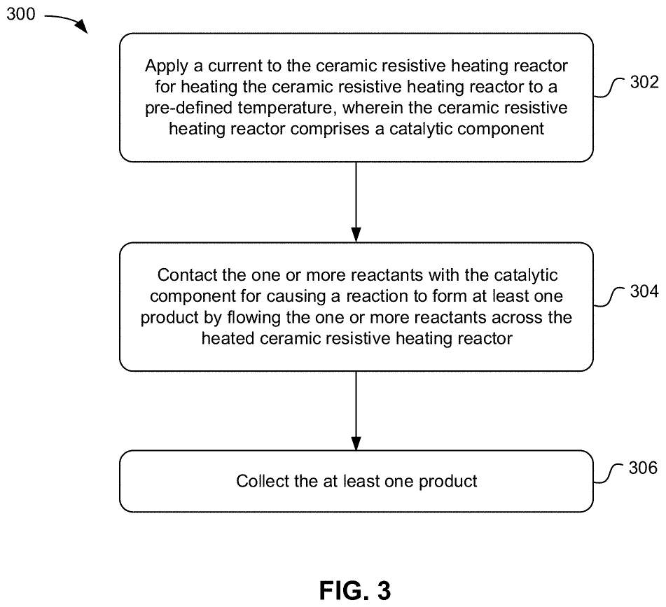

FIG. 3 shows a method 300 for using a ceramic resistive heating reactor for converting one or more reactants, in accordance with one aspect of one inventive concept. As an option, the present method 300 may be implemented to construct structures such as those shown in the other FIGS. described herein. Of course, however, this method 300 and others presented herein may be used to form structures for a wide variety of devices and/or purposes which may or may not be related to the illustrative embodiments listed herein. Further, the methods presented herein may be carried out in any desired environment. Moreover, more or less operations than those shown in FIG. 3 may be included in method 300, according to various embodiments. It should also be noted that any of the aforementioned features may be used in any of the embodiments described in accordance with the various methods.

Operation 302 includes applying a current to the ceramic resistive heating reactor for heating the ceramic resistive heating reactor to a pre-defined temperature, such as to a temperature within a range of operating temperatures. The ceramic resistive heating reactor includes a catalytic component, as described here. The pre-defined temperature may be in a range of greater than about 100° C. to about 1500° C. but may be higher or lower. The pre-defined temperature may be an effective temperature for a desired reaction of a contacting reactant to form at least one desired product. In various approaches, the temperature may be defined by the specific desired reaction, the reaction conditions, the catalyst, etc. The level of current to apply to the ceramic resistive heating reactor may be predetermined, adjusted periodically and/or in real time based at least in part on a temperature sensor reading, a chemical sensor reading, etc. In various approaches, the reactant may include a gas, a liquid, a gas dissolved in a liquid, a gas entrained in a liquid (e.g., gas bubbles in a liquid), a solid dissolved in a liquid, a solid entrained in a liquid (e.g., a slurry of a solid and a liquid), a solid fluidized in a gas, a liquid dissolved in a different liquid, or a combination thereof.

Operation 304 includes contacting the one or more reactants with the catalytic component for causing a reaction to form at least one product by flowing the one or more reactants (e.g., gas, liquid, etc.) across the heated ceramic resistive heating reactor. In one approach, a gas reactant may include CO₂. In another approach, a gas reactant may include one or more of the following: H₂, CH₄, etc. In one approach, a liquid reactant may include one or more of the following: a hydrocarbon, a carbohydrate, an organic molecule, etc. In another approach, a mixture of CO₂ and liquid H₂O reactants at >200° C. form carbonic acid which may serve as a relatively environmentally benign method of acid catalysis for the chemical processing industry.

Operation 306 includes collecting at least one product from the contacting of the reactant with the catalytic component. In some approaches, the collecting at least one product may include one or more of the following: collecting a solid product, collecting a liquid product, collecting a gas product.

In various approaches, depending on the desired reaction, the ceramic resistive heating reactor may be configured to form at least one of the following products: CO, CH₄, H₂O, H₂, O₂, etc.

In some approaches, the resistive heating reactor may be configured to separate gases as in a membrane reactor. For example, in an application of an oxygen recovery reaction of Equation 5 (above), the reactant gas CO₂ may be split into carbon monoxide (CO) and oxygen (O₂). The O₂ may be recovered on one side of the resistive heating element and CO may be collected on the other side of the resistive heating element.

In Use

Various aspects of an inventive concept described herein may be developed for Potential commercial uses for the material: construction of reactors for chemical transformations of molecules where the reactions require temperatures in excess of 100° C. Likely mostly useful for reactions that are endothermic and operate at temperatures in excess of 500° C. with gas phase reactants.

Government use would potentially be for military applications, for example where it is desired to convert CO₂ to CO on a deployed ship, and then use that CO to synthesize fuels for planes/other ships in the field. The presently-disclosed technology provides the first step to enable this.

Space programs may be interested in the oxygen recovery reaction as a way of getting O₂ back from CO₂ that astronauts exhale, for long term space flight missions and exoplanet exploration.

The inventive concepts disclosed herein have been presented by way of example to illustrate the myriad features thereof in a plurality of illustrative scenarios, aspects of an inventive concept, and/or implementations. It should be appreciated that the concepts generally disclosed are to be considered as modular, and may be implemented in any combination, permutation, or synthesis thereof. In addition, any modification, alteration, or equivalent of the presently disclosed features, functions, and concepts that would be appreciated by a person having ordinary skill in the art upon reading the instant descriptions should also be considered within the scope of this disclosure.

While various aspects of an inventive concept have been described above, it should be understood that they have been presented by way of example only, and not limitation. Thus, the breadth and scope of an aspect of an inventive concept of the present invention should not be limited by any of the above-described exemplary aspects of an inventive concept but should be defined only in accordance with the following claims and their equivalents.

What is claimed is:

1. A product, comprising:
a resistive heating reactor comprising:
a three-dimensional resistive heating element formed by additive manufacturing, wherein the resistive heating element has a pre-defined geometric arrangement of features, wherein the resistive heating element comprises a conductive ceramic material; and
a catalytic component on at least an external surface of the resistive heating element.

2. The product as recited in claim 1, wherein the catalytic component is integrated into the resistive heating element.

3. The product as recited in claim 1, wherein an amount of catalytic component is in a range of greater than 0.1 weight percent to less than 50 weight percent of total weight of the resistive heating element and the catalytic component.

4. The product as recited in claim 1, wherein the catalytic component is in a coating on only a portion of a surface of the resistive heating element.

5. The product as recited in claim 4, wherein a thickness of the coating is in a range of greater than 0 nanometers and less than 50 microns.

6. The product as recited in claim 4, wherein the coating on only the portion of the surface of the resistive heating element is at a pre-defined location.

7. The product as recited in claim 1, wherein the catalytic component is selected from the group consisting of: a metal, a metal oxide, a reducible metal oxide, a metal carbide, a perovskite, a zeolite, a metal organic framework (MOF), and a combination thereof.

8. The product as recited in claim 7, wherein the catalytic component is in nanoparticle form.

9. The product as recited in claim 1, wherein the melting point of the conductive ceramic material is greater than 1800 degrees Celsius.

10. The product as recited in claim 1, wherein the conductive ceramic material includes at least one material selected from the group consisting of: a metal carbide, a metalloid carbide, a metal boride, a metalloid boride, a metal oxide, a metalloid oxide, a metal nitride, a metalloid nitride, a metal silicide, and a combination thereof.

11. The product as recited in claim 1, wherein the pre-defined geometric arrangement of features comprises an open cell structure having continuous channels through the resistive heating element from one side of the resistive heating element to the other side of the resistive heating element.

12. The product as recited in claim 11, wherein the open cell structure is selected from the group consisting of: a Triply Periodic Minimal Structure (TPMS), a log-pile structure, a hollow cylinder, and a gyroid type structure.

13. A method of forming a product for high temperature conversion of one or more reactants, the method comprising:
fabricating a resistive heating reactor using, at least in part, additive manufacturing, wherein the resistive heating reactor comprises a conductive ceramic material and a catalytic component.

14. The method as recited in claim 13, wherein fabricating the resistive heating reactor comprises,
forming a resistive heating element; and
coating the resistive heating element with the catalytic component.

15. The method as recited in claim 14, wherein the coating forms a layer of catalytic component directly on a surface of the resistive heating element, wherein a thickness of the layer is in a range of greater than 0 nanometers to less than 50 microns.

16. The method as recited in claim 13, wherein fabricating the resistive heating reactor comprises,
printing a resistive heating element using one or more inks.

17. The method as recited in claim 13, wherein fabricating the resistive heating reactor comprises,
obtaining an already manufactured resistive heating element; and
printing the catalytic component onto a surface of the already manufactured resistive heating element.

18. The method as recited in claim 13, wherein the catalytic component is selected from the group consisting of: a metal, a metal oxide, a reducible metal oxide, a metal carbide, a perovskite, a zeolite, a metal organic framework (MOF), and a combination thereof.

19. The method as recited in claim 13, wherein the melting point of the conductive ceramic material is greater than 1800 degrees Celsius.

20. The method as recited in claim 13, wherein the conductive ceramic material includes at least one material selected from the group consisting of: a metal carbide, a metalloid carbide, a metal boride, a metalloid boride, a metal oxide, a metalloid oxide, a metal nitride, a metalloid nitride, a metal silicide, and a combination thereof.

21. The method as recited in claim 13, wherein the additive manufacturing technique includes a technique selected from the group consisting of: an extrusion-based technique, a powder bed-based technique, a material jetting technique, a sheet lamination technique, an electrostatic deposition technique, a laser fusion technique, use of a mold, and use of a template.

22. A method of using a ceramic resistive heating reactor for converting one or more reactants, the method comprising:
applying a current to the ceramic resistive heating reactor for heating the ceramic resistive heating reactor to a pre-defined temperature, wherein the ceramic resistive heating reactor comprises a catalytic component;
contacting the one or more reactants with the catalytic component for causing a reaction to form at least one product by flowing the one or more reactants across the heated ceramic resistive heating reactor; and
collecting the at least one product.

23. The method as recited in claim 22, wherein the pre-defined temperature is in a range of greater than about 100 degrees Celsius to about 1500 degrees Celsius.

24. The method as recited in claim 22, wherein at least one of the one or more reactants is selected from the group consisting of: a gas, a liquid, a solid dissolved in a liquid, a first liquid dissolved in a second liquid, a gas dissolved in a liquid, a gas entrained in a liquid, a solid entrained in a liquid, a solid fluidized in a gas, and a combination thereof.

25. The method as recited in claim 22, wherein the ceramic resistive heating reactor is configured to form at least one product selected from the group consisting of: CO, CH₄, H₂O, H₂, and O₂.
